(19) United States
(12) Patent Application Publication
(10) Pub. No.: US 2017/0172963 A1
(43) Pub. Date: Jun. 22, 2017

(54) COMPOSITIONS AND METHODS FOR THE PREVENTION AND OR TREATMENT OF SCHISTOSOMIASIS

(71) Applicants: Kevin HADLEY, Elkridge, MD (US); Rashika ELRIDI, Dokki, Giza (EG); DSM IP ASSETS B.V., Te Heerlen (NL)

(72) Inventors: Kevin HADLEY, Elkridge, MD (US); Rashiki EL RIDI, Dokki (EG)

(73) Assignee: DSM IP ASSETS B.V., TE Heerlen (NL)

(21) Appl. No.: 15/118,190

(22) PCT Filed: Feb. 13, 2015
(86) PCT No.: PCT/US15/15728

§ 371 Date: Aug. 11, 2016

Related U.S. Application Data
(60) Provisional application No. 61/940,041, filed on Feb. 14, 2014.

(51) Int. Cl. A61K 31/202; A61K 31/4985

(52) U.S. Cl. CPC A61K 31/202; A61K 31/4985; A61K 2300/00

(57) ABSTRACT

This invention relates to compositions for the prevention and treatment of schistosomiais comprising a polyunsaturated fatty acid and a therapeutic agent. The invention further relates to methods for the prevention and treatment of schistosomiasis, methods to confer resistance to schistosomiasis and methods to prevent re-occurrence of schistosomiasis comprising administration of a polyunsaturated fatty acid and a therapeutic agent.

### CROSS-REFERENCE TO RELATED APPLICATIONS

This application claims the benefit of the filing date of U.S. Provisional Patent Application No. 61/940,041 filed Feb. 14, 2014, the disclosure of which is hereby incorporated herein by reference.

### FIELD OF THE INVENTION

Disclosed herein are compositions for the prevention and treatment of schistosomiais comprising a polyunsaturated fatty acid and a therapeutic agent. Further disclosed herein are methods for the prevention and treatment of schistosomiasis, methods to confer resistance to schistosomiasis and methods to prevent re-occurrence of schistosomiasis comprising administration of a polyunsaturated fatty acid and a therapeutic agent.

### BACKGROUND OF THE INVENTION

Schistosomiasis (also known as bilharzia, bilharziosis or snail fever) is a severe parasitic disease caused by several species of fluke of the genus Schistosoma. The disease is found predominantly in Asia, Africa and South America and especially in areas with water that are contaminated with snails that carry the parasite. It is the second most common parasitic disease after malaria and affects mainly children. Schistosomiasis is caused by the platyhelminth worms of the genus Schistosoma, trematodes that live in the bloodstream of humans and animals. Three species (Schistosoma mansoni, Schistosoma haematobium and Schistosoma japonicum) account for the majority of the infections. Schistosomes migrate against the direction of blood flow to their permanent abode with S. masoni and S. japonicum to the inferior mesoteric and S. haematobium to the peri-vesical venous plexus. Schistosomes lay eggs near the conduit for egg passage to the external environment to complete the life cycle. Accordingly, massive number of eggs exit daily from the blood capillaries to the lumen of the gut or urinary bladder whereby they may be detected by analysis of stool and urine samples.

Arachidonic acid (ARA), all-cis 5,8,11,14-eicosatetreanoic acid, an omega-6 fatty acid: 20:4(ω-6), is present in the phospholipids of membranes of the body's cells and has been hypothesized to act to kill juvenile and adult male and female S. masoni and S. haematobium worms.

Praziquantel (PZQ) is currently the only drug for treatment of Schistosomiasis, particularly S. masoni. However, the rate of failure of cure in children following treatment was consistently associated with high intensity of infection, requiring repeated treatments and accelerating the impending threat of schistosomes' resistance to the drug. Further, PZQ was not shown to confer resistance to schistosomes. As a result, there is a need for a composition, alone or in combination with PZQ, for the treatment and/or prevention of Schistosomiasis and to confer resistance to and/or prevent re-occurrence of schistosomiasis.

### SUMMARY OF THE INVENTION

The present invention relates to compositions and methods for the prevention and treatment of schistosomiasis. The present invention also relates to methods to confer resistance to and/or prevent re-occurrence of schistosomiasis.

In a preferred embodiment, the composition comprises a polyunsaturated fatty acid (PUFA) and a therapeutic agent. Preferably, the composition comprises a PUFA that is an omega-6 fatty acid. More preferably, the composition comprises a PUFA that is an omega-6 fatty acid that is all-cis 5,8,11,14-eicosatetreanoic acid, also known as arachidonic acid (ARA). In a preferred embodiment, the therapeutic agent is 2-(Cyclohexylcarbonyl)-1,2,3,6,7,11b-hexahydro-4H-pyrazino[2,1-c]isoquinolin-4-one, also known as Praziquantel (PZQ), or a derivative thereof.

In one embodiment, the methods disclosed herein comprise administration of a polyunsaturated fatty acid (PUFA) and a therapeutic agent. In one embodiment, the PUFA is administered in one dosage and the therapeutic agent is administered in a second dosage. In another embodiment, the PUFA and the therapeutic agent are administered in the same dosage. Preferably, the PUFA is an omega-6 fatty acid. More preferably, the PUFA is an omega-6 fatty acid that is all-cis 5,8,11,14-eicosatetreanoic acid, also known as arachidonic acid (ARA). In a preferred embodiment, the therapeutic agent is 2-(Cyclohexylcarbonyl)-1,2,3,6,7,11b-hexahydro-4H-pyrazino[2,1-c]isoquinolin-4-one, also known as Praziquantel (PZQ), or a derivative thereof.

### DETAILED DESCRIPTION OF THE INVENTION

Disclosed herein are compositions for the prevention and/or treatment of schistosomiais comprising a polyunsaturated fatty acid and a therapeutic agent. Further disclosed herein are methods for the prevention and/or treatment of schistosomiasis, methods to confer resistance to schistosomiasis and methods to prevent re-occurrence of schistosomiasis comprising administration of a polyunsaturated fatty acid and a therapeutic agent in a therapeutically effective amount.

The features and advantages of the invention may be more readily understood by those of ordinary skill in the art upon reading the following detailed description. It is to be appreciated that certain features of the invention that are, for clarity reasons, described above and below in the context of separate embodiments, may also be combined so as to sub-combinations thereof.

Embodiments identified herein as exemplary are intended to be illustrative and not limiting.

The term “about” is intended to capture variations above and below the stated number that may achieve substantially the same results as the stated number.

Fatty acids are classified based on the length and saturation characteristics of the carbon chain. Fatty acids present in a microbial oil can have from 4 to 28 carbon atoms and are termed short chain, medium chain, or long chain fatty acids based on the number of carbons present in the chain. Fatty acids are termed saturated fatty acids when no double bonds are present between the carbon atoms, and are termed unsaturated fatty acids when double bonds are present. Unsaturated long chain fatty acids are monounsaturated when only one double bond is present and are polyunsaturated when more than one double bond is present.

Polyunsaturated fatty acids (PUFAs) are classified based on the position of the first double bond from the methyl end of the fatty acid; omega-3 (n-3) fatty acids contain a first double bond at the third carbon, while omega-6 (n-6) fatty acids contain a first double bond at the sixth carbon. For example, docosahexaenoic acid (DHA) is an omega-3 long chain polyunsaturated fatty acid (LC-PUFA) with a chain length of 22 carbons and 6 double bonds, often designated as “22:6n-3.” In one embodiment, the PUFA is selected from an omega-3 fatty acid, an omega-6 fatty acid, and mixtures thereof. In another embodiment, the PUFA is selected from long-chain polyunsaturated fatty acids (LC-PUFAs). In a still further embodiment, the PUFA is selected from docosahexaenoic acid (DHA), eicosapentaenoic acid (EPA), docosapentaenoic acid (DPA), arachidonic acid (ARA), gamma-linolenic acid (GLA), dihomo-gamma-linolenic acid (DGLA), stearidonic acid (SDA), and mixtures thereof. In another embodiment, the PUFA is selected from DHA, ARA, and mixtures thereof. In a further embodiment, the PUFA is DHA. In yet a further embodiment, the PUFA is ARA.

LC-PUFAs are fatty acids that contain at least 3 double bonds and have a chain length of 18 or more carbons or 20 or more carbons. LC-PUFAs of the omega-6 series include, but are not limited to, di-homo-gammalinoleic acid (C20:3n-6), arachidonic acid (C20:4n-6) (“ARA”), docosatetraenoic acid or adrenic acid (C22:4n-6), and docosapentaenoic acid (C22:5n-6) (“DPA n-6”). The LC-PUFAs of the omega-3 series include, but are not limited to, eicosatrienoic acid (C20:3n-3), eicosatetraenoic acid (C20:4n-3), eicosapentaenoic acid (C20:5n-3) (“EPA”), docosapentaenoic acid (C22:5n-3), and docosahexaenoic acid (C22:6n-3). The LC-PUFAs also include fatty acids with greater than 22 carbons and 4 or more double bonds including, but not limited to, C24:6(n-3) and C28:8(n-3).

The PUFAs can be in the form of a free fatty acid, salt, fatty acid ester (e.g. methyl or ethyl ester), monoacylglycerol (MAG), diacylglycerol (DAG), triacylglycerol (TAG), and/or phospholipid (PL).

Any source of PUFA can be used in the compositions and methods of the invention, including, for example, animal, plant and microbial sources. Preferred polyunsaturated fatty acid (PUFA) sources can be any source of PUFA that are suitable for use in the present invention.

Examples of animal sources include aquatic animals (e.g., fish, marine mammals, crustaceans, rotifers, etc.). Examples of plant sources include microalgae, flaxseeds, rapeseeds, corn, evening primrose and borage. Examples of microorganisms include microalgae, protists, bacteria and fungi (including yeast). The use of a microorganism source, such as microalgae, can provide organoleptic advantages.

In accordance with the present invention, the polyunsaturated fatty acids that are used in the compositions described herein are in a variety of forms, for example, such forms include, but are not limited to: a highly purified algal oil comprising a PUFA, a plant oil comprising the PUFA, triglyceride oil comprising the PUFA, phospholipid comprising the PUFA, a combination of protein and phospholipids comprising the PUFA, dried marine microalgae comprising the PUFA, sphingolipids comprising the PUFA, esters of the PUFA, free fatty acid, a conjugate of the PUFA with another bioactive molecule, and conjugates thereof.

In one embodiment, the composition comprises a polyunsaturated fatty acid (PUFA) and a therapeutic agent. Preferably, the composition comprises a PUFA that is an omega-6 fatty acid. More preferably, the composition comprises a PUFA that is an omega-6 fatty acid that is all-cis 5,8,11,14-eicosatetreanoic acid, also known as arachidonic acid (ARA). In one embodiment, the composition comprises a therapeutic agent that is a trematocide. Preferably, the composition comprises a therapeutic agent that is a trematocide that is 2-(Cyclohexylcarbonyl)-1,2,3,6,7,11b-hexahydro-4H-pyrazino[2,1-a] isoquinolin-4-one, also known as Praziquantel (PZQ), or a derivative thereof.

In some embodiments, the PUFA is administered in one dosage and the therapeutic compound is administered in a second dosage.

In some embodiments, the PUFA is administered in an amount of about 5 mg/kg body weight/day, about 7 mg/kg body weight/day, about 10 mg/kg body weight/day, about 12 mg/kg body weight/day, about 15 mg/kg body weight/day, or from about 5 to about 15 mg/kg body weight/day, from about 7 to about 12 mg/kg body weight/day, from about 9 to about 11 mg/kg body weight/day.

In one embodiment, the PUFA is administered in an amount of about 10 mg/kg body weight/day.

In some embodiments, the PUFA is administered in an amount of from about 10 mg/kg body weight/day to about 600 mg/kg body weight/day, from about 20 mg/kg body weight/day to about 550 mg/kg body weight/day, from about 40 mg/kg body weight/day to about 500 mg/kg body weight/day, from about 50 mg/kg body weight/day to about 450 mg/kg body weight/day, from about 60 mg/kg body weight/day to about 400 mg/kg body weight/day, from about 70 mg/kg body weight/day to about 350 mg/kg body weight/day, from about 80 mg/kg body weight/day to about 300 mg/kg body weight/day, from about 90 mg/kg body weight/day to about 250 mg/kg body weight/day, from about 100 mg/kg body weight/day to about 200 mg/kg body weight/day, from about 110 mg/kg body weight/day to about 150 mg/kg body weight/day, about 10 mg/kg body weight/day about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, about 100 mg/kg body weight/day, about 110 mg/kg body weight/day, about 120 mg/kg body weight/day, about 130 mg/kg body weight/day, about 140 mg/kg body weight/day, about 150 mg/kg body weight/day, about 160 mg/kg body weight/day, about 170 mg/kg body weight/day, about 180 mg/kg body weight/day, about 190 mg/kg body weight/day, about 200 mg/kg body weight/day, about 250 mg/kg body weight/day, about 300 mg/kg body weight/day, about 350 mg/kg body weight/day, about 400 mg/kg body weight/day, about 450 mg/kg body weight/day, about 500 mg/kg body weight/day, about 550 mg/kg body weight/day, about 600 mg/kg body weight/day.

In one embodiment, the PUFA is administered in an amount of about 150 mg/kg body weight/day.

In some embodiments, the PUFA is administered in a single dose or multiple doses.

In some embodiments, the PUFA is administered over about 30 days, over about 20 days, over about 15 days, over about 10 days, over about 5 days, or over about 1 to 30 days, over about 1 to 20 days, over about 1 to 15 days, over about 1 to 10 days, over about 1 to 5 days.

In some embodiments, the therapeutic agent is administered in an amount of about 20 mg/kg body weight/day, in an amount of about 25 mg/kg body weight/day, in an amount of about 30 mg/kg body weight/day, in an amount of about 35 mg/kg body weight/day, in an amount of about 40 mg/kg body weight/day, in an amount of about 45 mg/kg body weight/day, in an amount of from about 50 mg/kg body weight/day, in an amount of about 55 mg/kg body weight/day, in an amount of about 60 mg/kg body weight/day, in an amount of from about 20 to about 60 mg/kg body weight/day, in an amount of from about 25 to about 55 mg/kg body weight/day, in an amount of from about 30 to about 50 mg/kg body weight/day, in an amount of from about 35 to about 45 mg/kg body weight/day.

In one embodiment, the therapeutic agent is administered in an amount of about 40 mg/kg body weight/day.

In some embodiments, the Schistosomiasis is Schistosoma mansoni, Schistosoma haematobium and Schistosoma japonicum. In some embodiments, the Schistosomiasis is Schistosoma mansoni.

In one embodiment, the methods disclosed herein comprise administration of a polyunsaturated fatty acid (PUFA) and a therapeutic agent for the treatment or prevention of schistostomiasis. In another embodiment, the methods disclosed herein comprise administration of a polyunsaturated fatty acid and a therapeutic agent to confer resistance to schistostomiasis. In another embodiment, the methods disclosed herein comprise administration of a polyunsaturated fatty acid and a therapeutic agent to prevent re-occurrence of schistosomiasis.

In some embodiments, the PUFA is an omega-6 fatty acid. More preferably, the PUFA is an omega-6 fatty acid that is all-cis 5,8,11,14-eicosatetreanoic acid, also known as arachidonic acid (ARA).

In some embodiments, the therapeutic agent is a trematocide. Preferably, the therapeutic agent that is a trematocide is 2-(Cyclohexylcarbonyl)-1,2,3,6,7,11b-hexahydro-4H-pyrazino[2,1-c]isoquinolin-4-one, also known as Praziquantel (PZQ), or a derivative thereof.

In some embodiments, the PUFA is administered in one dosage and the therapeutic compound is administered in a second dosage.

In some embodiments, the PUFA is administered in an amount of from about 5 mg/kg body weight/day, about 7 mg/kg body weight/day, about 10 mg/kg body weight/day, about 12 mg/kg body weight/day, about 15 mg/kg body weight/day or from about 5 to about 15 mg/kg body weight/day, from about 7 to about 12 mg/kg body weight/day, from about 9 to about 11 mg/kg body weight/day.

In one embodiment, the PUFA is administered in an amount of about 10 mg/kg body weight/day.

In some embodiments, the PUFA is administered in an amount of from about 10 mg/kg body weight/day to about 600 mg/kg body weight/day, from about 20 mg/kg body weight/day to about 550 mg/kg body weight/day, from about 40 mg/kg body weight/day to about 500 mg/kg body weight/day, from about 50 mg/kg body weight/day to about 450 mg/kg body weight/day, from about 60 mg/kg body weight/day to about 400 mg/kg body weight/day, from about 70 mg/kg body weight/day to about 350 mg/kg body weight/day, from about 80 mg/kg body weight/day to about 300 mg/kg body weight/day, from about 90 mg/kg body weight/day to about 250 mg/kg body weight/day, from about 100 mg/kg body weight/day to about 200 mg/kg body weight/day, from about 110 mg/kg body weight/day to about 150 mg/kg body weight/day, about 10 mg/kg body weight/day about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, about 100 mg/kg body weight/day, about 110 mg/kg body weight/day, about 120 mg/kg body weight/day, about 130 mg/kg body weight/day, about 140 mg/kg body weight/day, about 150 mg/kg body weight/day, about 160 mg/kg body weight/day, about 170 mg/kg body weight/day, about 180 mg/kg body weight/day, about 190 mg/kg body weight/day, about 200 mg/kg body weight/day, about 250 mg/kg body weight/day, about 300 mg/kg body weight/day, about 350 mg/kg body weight/day, about 400 mg/kg body weight/day, about 450 mg/kg body weight/day, about 500 mg/kg body weight/day, about 550 mg/kg body weight/day, about 600 mg/kg body weight/day.

In one embodiment, the PUFA is administered in an amount of about 150 mg/kg body weight/day.

In some embodiments, the PUFA is administered in a single dose or multiple doses.

In some embodiments, the PUFA is administered over about 30 days, over about 20 days, over about 15 days, over about 10 days, over about 5 days, over about 1 to 30 days, over about 1 to 20 days, over about 1 to 15 days, over about 1 to 10 days, over about 1 to 5 days.

In some embodiments, the therapeutic agent is administered in an amount of about 20 mg/kg body weight/day, in an amount of about 25 mg/kg body weight/day, in an amount of about 30 mg/kg body weight/day, in an amount of about 35 mg/kg body weight/day, in an amount of about 40 mg/kg body weight/day, in an amount of about 45 mg/kg body weight/day, in an amount of about 50 mg/kg body weight/day, in an amount of about 55 mg/kg body weight/day, in an amount of about 60 mg/kg body weight/day, in an amount of from about 20 to about 60 mg/kg body weight/day, in an amount of from about 25 to about 55 mg/kg body weight/day, in an amount of from about 30 to about 50 mg/kg body weight/day, in an amount of from about 35 to about 45 mg/kg body weight/day.

In one embodiment, the therapeutic agent is administered in an amount of about 40 mg/kg body weight/day.

In some embodiments, the Schistosomiasis is Schistosoma mansoni, Schistosoma haematobium and Schistosoma japonicum. In some embodiments, the Schistosomiasis is Schistosoma mansoni.

### EXAMPLES

### Example 1

### Subjects

Approximately 2000 school children in villages of three districts in the Menoufiya governorate located about 100 km north of Cairo, Egypt were screened. Two microscopic slides of stool samples were examined for each child on 3 consecutive days by the Kato-Katz method and egg counts per gram (epg) were recorded. 66 children, 44 males and 22 females, 6-15 years old (mean±SD=11.7±1.6), with weight range of 25-52 kg (mean±SD=37.8±8.6), and epg of 24-384 were selected as test subjects. 20 sex, age, and social conditions-matched, parasite-free children were used as controls.

### Treatment

The 66 children were divided randomly into three groups whereby the first group of 20 children were given a single oral dose of Praziquantel (PZQ) (40 mg/kg body weight) on the first day of treatment and a placebo was given for the next three weeks (5 doses/week) (“PZQ-Placebo Treatment”). The PZQ was Distocide (Epico, El-Asher-Men-Ramadan City, Egypt). The placebo was 1 g VegCap, containing corn-soy (DSM Nutritional Products, Columbia, Md.). The second group of 23 children received ARA (10 mg/kg body weight/day) for 15 days over three weeks (5 days/week) (“ARA Treatment”) The ARA was 1 g VegCap (DSM) containing approximately 395 mg ARA/capsule. The third group of 23 children were given Praziquantel (40 mg/kg body weight) in the first day of treatment and then received 15 doses of ARA (10 mg/kg/day; 5 doses/week) (“PZQ+ARA Treatment”).

### Study Design

Approximately 10 ml blood were obtained from each child 2-3 days before the start of the treatment and 3 days after the end of the ARA (or ARA+PZQ) treatment, corresponding to 18 days after PZQ-only treatment. Stool samples were obtained from each child on 3 consecutive days 1 week after the end of the ARA treatment (corresponding to 4 weeks after the PZQ-only treatment) and 6 weeks after the end of the ARA treatment (corresponding to 9 weeks after the PZQ-only treatment). Epg was assessed on each stool sample.

### Biochemical and Hematological Parameters

Samples for biochemical analysis were put in a plain tube and allowed to clot for at least 60 minutes, centrifuged, and the serum was collected. Two ml blood samples were dispensed into a tube with EDTA and analyzed by automated blood hematology analyzer (XT 1800i, Sysmex Corporation).

### Levels of Plasma Interleukin-10 (IL-10) and Interferon-Gamma (IFN-γ)

Plasma was retrieved from heparinized blood following centrifugation at 400 g for 20 minutes and stored at −76° C. until assayed by capture enzyme-linked immunosorbent assay (ELISA) for levels of IL-10 and IFN-γ (ELISA MAX™ Set, BioLegend, San Diego, Calif.) according to the manufacturer's protocol.

### Whole Blood Cytokine Response to Parasite Antigens

Heparinized whole blood cells were diluted 1:4 in Roswell Park Memorial Institute (RPMI)-1640 medium supplemented with 200 U/ml penicillin, 200 μg/mL streptomycin, 50 μg/mL amphotericin, and 20 μg/mL polymixin B (Sigma) as an inhibitor of any residual lipopolysaccharide contamination of antigens. 200 μl diluted blood was incubated in duplicate wells of sterile round-bottomed well microtiter plates with 50 μl medium containing 0 or 40 μg/mL recombinant S. mansoni glyceraldehyde 3-phosphate dehydrogenase (rSG3PDH). Whole blood cultures were incubated for 72 hours at 37° C./3% CO₂, and then centrifuged at 400 g for 10 minutes. The cell-free supernatants were transferred into wells of sterile plate, and stored at −76° C. until assayed by capture ELISA for levels of released IL-4, IL-17 and IFN-γ (ELISA MAX™ Set, BioLegend, San Diego, Calif.) according to the manufacturer's protocol.

### Results

Subjects having initial baseline epg counts <100 were classified as having low infection. Subjects having initial baseline epg counts >100 but <400 were classified as having moderate infection. Subjects having initial baseline epg counts >400 were classified as having high infection.

All treatment groups had a % cure rate in the moderate infection group less than in the low infection group. The % cure for the PZQ-Placebo moderate and high infection group was 67% as compared to 71% for the low infection group. The % cure for the ARA moderate and high infection group was 50% as compared to 77% for the low infection group. The % cure rate for the PZQ-ARA moderate and high infection group was 71% as compared to 94% for the low infection group. The PZQ-ARA treatment group resulted in a higher % cure rate for the low infection groups versus the PZQ-Placebo treatment group or the ARA treatment group (94%, 71% and 77%, respectively). The % cure rate for the PZQ-ARA treatment group resulted in a higher % cure rate for the moderate and high infection groups versus the PZQ-Placebo treatment group or the ARA treatment group (71%, 67% and 50%, respectively).

### Efficacy of PZQ-Placebo Treatment

TABLE 1
PZQ-Placebo Treatment Subjects with initial baseline epg < 100

| Subject | Baseline | epg After | epg After |
|---|---|---|---|
| Number | epg | Week 4 | Week 9 |
| 1 | 96 | 0 | 0 |
| 4 | 24 | 0 | 0 |
| 7 | 24 | 5 | 0 |
| 10 | 48 | 5 | 20 |
| 13 | 48 | — | 10 |
| 16 | 24 | 8 | 4 |
| 19 | 48 | 0 | 0 |
| 34 | 72 | 0 | 0 |
| 37 | 24 | 0 | 0 |
| 40 | 48 | 0 | 6 |
| 46 | 48 | 0 | 0 |
| 54 | 96 | 0 | 0 |
| 66 | 96 | 0 | 0 |
| 58 | 48 | — | 0 |

As shown in Table 1, the PZQ-Placebo Treatment resulted in a 71% cure rate for children having low infection (10/14 children exhibited 0 epg after 9 weeks).

TABLE 2
PZQ- Placebo Treatment Subjects with initial baseline epg > 100

| Subject | Baseline | epg at | epg at |
|---|---|---|---|
| Number | epg | Week 4 | Week 9 |
| 22 | 120 | 0 | 10 |
| 25 | 192 | 16 | 0 |
| 28 | 120 | — | 30 |
| 31 | 168 | 0 | 0 |
| 43 | 3312 | — | 0 |
| 51 | 192 | 0 | 0 |

As shown in Table 2, the PZQ-Placebo Treatment resulted in a 67% cure rate for children having moderate and high infection (4/6 children exhibited 0 epg after 9 weeks).

Efficacy of ARA Treatment

TABLE 3
ARA Treatment Subjects with initial baseline epg < 100

| Subject | Baseline | epg After | epg After |
|---|---|---|---|
| Number | epg | Week 4 | Week 9 |
| 5 | 96 | 0 | 42 |
| 8 | 48 | 19 | 0 |
| 11 | 24 | 24 | 0 |
| 14 | 24 | 0 | 0 |
| 17 | 24 | 12 | 0 |
| 32 | 72 | — | 0 |
| 35 | 96 | 0 | 0 |
| 41 | 96 | 144 | 376 |
| 47 | 96 | 24 | 20 |
| 49 | 48 | 20 | 0 |
| 68 | 48 | 0 | 0 |
| 59 | 96 | 52 | 0 |
| 57 | 96 | 48 | 0 |

As shown in Table 3, the ARA Treatment resulted in a 77% cure rate for children having low infection (10/13 children exhibited 0 epg after week 9).

TABLE 4
ARA Treatment Subjects with initial baseline epg > 100

| Subject | Baseline | epg After | epg After |
|---|---|---|---|
| Number | epg | Week 4 | Week 9 |
| 2 | 144 | 96 | 48 |
| 20 | 192 | 48 | 28 |
| 23 | 192 | 60 | 20 |
| 26 | 168 | 96 | 152 |
| 29 | 384 | 240 | 0 |
| 38 | 312 | 96 | 0 |
| 44 | 912 | 749 | 0 |
| 52 | 286 | 216 | 108 |
| 55 | 192 | 84 | 0 |
| 62 | 120 | 0 | 0 |

As shown in Table 4, the ARA Treatment resulted in a 50% cure rate for children having moderate and high infection (5/10).

Efficacy of PZQ-ARA Treatment

TABLE 5
PZQ-ARA Treatment Subjects with initial baseline epg < 100

| Subject | Baseline | epg After | epg After |
|---|---|---|---|
| Number | epg | Week 4 | Week 9 |
| 3 | 48 | 0 | 0 |
| 6 | 96 | 0 | 0 |
| 9 | 72 | 0 | 0 |
| 12 | 24 | 0 | 0 |
| 21 | 72 | 0 | 38 |
| 33 | 96 | 0 | 0 |
| 36 | 24 | 0 | 0 |
| 39 | 96 | 12 | 0 |
| 48 | 96 | 0 | 0 |
| 50 | 72 | 24 | 0 |
| 53 | 96 | 0 | 0 |
| 67 | 72 | 0 | 0 |
| 56 | 96 | 0 | 0 |
| 60 | 48 | 0 | 0 |
| 63 | 96 | 0 | 0 |
| 65 | 72 | 0 | 0 |

As shown in Table 5, the PZQ-ARA Treatment resulted in a 94% cure rate for children having low infection (15/16 children exhibited 0 epg after week 9).

TABLE 6
PZQ-ARA Treatment Subjects with initial baseline epg > 100

| Subject | Baseline | epg After | epg After |
|---|---|---|---|
| Number | epg | Week 4 | Week 9 |
| 15 | 384 | 248 | 0 |
| 18 | 192 | 88 | 0 |
| 24 | 192 | 0 | 0 |
| 27 | 1800 | 102 | 40 |
| 30 | 672 | 0 | 0 |
| 42 | 288 | 0 | 10 |
| 45 | 120 | 29 | 0 |

As shown in Table 6, the PZQ-ARA Treatment resulted in a 71% cure rate for children having moderate and high infection (5/7 children exhibited 0 epg after week 9).

### Example 2

### Subjects

School children from El Kafr Sheikh were screened similar to those school children in Example 1 above.

### Treatment

The children were divided randomly into three groups whereby the first group of children were given a single oral dose of Praziquantel (PZQ) (40 mg/kg body weight) on the first day of treatment and a placebo was given for the next three weeks (5 doses/week) (“PZQ-Placebo Treatment”). The PZQ was Distocide (Epico, El-Asher-Men-Ramadan City, Egypt). The placebo was 1 g VegCap, containing corn-soy (DSM Nutritional Products, Columbia, Md.). The second group of children received ARA (10 mg/kg body weight/day) for 15 days over three weeks (5 days/week) (“ARA Treatment”). The ARA was 1 g VegCap (DSM) containing approximately 395 mg ARA/capsule. The third group of children were given Praziquantel (40 mg/kg body weight) in the first day of treatment and then received 15 doses of ARA (10 mg/kg/day; 5 doses/week) (“PZQ-ARA Treatment”).

### Study Design

Approximately 10 ml blood were obtained from each child 2-3 days before the start of the treatment and 3 days after the end of the ARA (or ARA+PZQ) treatment, corresponding to 18 days after PZQ-only treatment. Stool samples were obtained from each child on 3 consecutive days 1 week after the end of the ARA treatment (corresponding to 4 weeks after the PZQ-only treatment) and 6 weeks after the end of the ARA treatment (corresponding to 9 weeks after the PZQ-only treatment). Epg was assessed on each stool sample.

### Biochemical and Hematological Parameters

Samples for biochemical analysis were put in a plain tube and allowed to clot for at least 60 minutes, centrifuged, and the serum was collected. Two ml blood samples were dispensed into a tube with EDTA and analyzed by automated blood hematology analyzer (XT 1800i, Sysmex Corporation).

### Levels of Plasma Interleukin-10 (IL-10) and Interferon-Gamma (IFN-γ)

Plasma was retrieved from heparinized blood following centrifugation at 400 g for 20 minutes and stored at −76° C. until assayed by capture enzyme-linked immunosorbent assay (ELISA) for levels of IL-10 and IFN-γ (ELISA MAX™ Set, BioLegend, San Diego, Calif.) according to the manufacturer's protocol.

### Whole Blood Cytokine Response to Parasite Antigens

Heparinized whole blood cells were diluted 1:4 in Roswell Park Memorial Institute (RPMI)-1640 medium supplemented with 200 U/ml penicillin, 200 μg/mL streptomycin, 50 μg/mL amphotericin, and 20 μg/mL polymixin B (Sigma) as an inhibitor of any residual lipopolysaccharide contamination of antigens. 200 n1 diluted blood was incubated in duplicate wells of sterile round-bottomed well microtiter plates with 50 n1 medium containing 0 or 40 μg/mL recombinant S. mansoni glyceraldehyde 3-phosphate dehydrogenase (rSG3PDH). Whole blood cultures were incubated for 72 hours at 37° C./3% CO₂, and then centrifuged at 400 g for 10 minutes. The cell-free supernatants were transferred into wells of sterile plate, and stored at −76° C. until assayed by capture ELISA for levels of released IL-4, IL-17 and IFN-γ (ELISA MAX™ Set, BioLegend, San Diego, Calif.) according to the manufacturer's protocol.

### Results

Subjects having initial baseline epg counts <100 were classified as having low infection. Subjects having initial baseline epg counts >100 but <400 were classified as having moderate infection. Subjects having initial baseline epg counts >400 were classified as having high infection.

All treatment groups had a % cure rate in the high infection group less than either the moderate infection group or the low infection group. All treatment groups had a % cure rate in the moderate infection group less than the low infection group.

The % cure rate for the PZQ-ARA treatment group resulted in a higher % cure rate for the low infection group versus the PZQ-Placebo treatment group or the ARA treatment group (74%, 57% and 50%, respectively). See Table 7.

The % cure rate for the PZQ-ARA treatment group resulted in a higher % cure rate for the moderate infection group versus the PZQ-Placebo treatment group or the ARA treatment group (73%, 70% and 49%, respectively). See Table 8.

The % cure rate for the PZQ-ARA treatment group resulted in a higher % cure rate for the high infection group versus the PZQ-Placebo treatment group or the ARA treatment group (84%, 81% and 65%, respectively). See Table 9.

Decreased levels of circulating IL-10 and IFN-γ were shown in all treatment groups. Treatment with ARA alone or combined with PZQ was more effective in altering the immune responses of children towards reducing production of Il-10 and IFN-γ. These significant decreases in the levels of immunosuppressive IL-10 and IFN-γ indicate a resistance to reinfection with schistosomes. See Tables 10 and 11.

TABLE 7
Effect of Treatment with PZQ, ARA or PZQ + ARA
Subjects with initial baseline epg < 100

| | Number of | Baseline | | % epg |
|---|---|---|---|---|
| Treatment | Children | mean epg | % cured | reduction* |
| PZQ | 35 | 38.7 | 57 (20/35) | 50 |
| ARA | 43 | 34.2 | 50 (22/43) | −2.6 |
| PZQ + ARA | 39 | 41.3 | 74 (29/39) | 52 |

*% epg reduction = ((mean baseline epg − mean epg 6 weeks after treatment)/mean baseline epg) * 100

TABLE 8
Effect of Treatment with PZQ, ARA or PZQ + ARA Subjects
with initial baseline <400 epg > 100

| | Number of | Baseline | | % epg |
|---|---|---|---|---|
| Treatment | Children | mean epg | % cured | reduction* |
| PZQ | 23 | 226.1 | 43 (10/23) | 70 |
| ARA | 29 | 195.6 | 28 (4/19) | 49 |
| PZQ + ARA | 26 | 215.8 | 73 (19/26) | 73 |

*% epg reduction = ((mean baseline epg − mean epg 6 weeks after treatment)/mean baseline epg) * 100

TABLE 9
Effect of Treatment with PZQ, ARA or PZQ + ARA Subjects
with initial baseline epg > 400 epg

| | Number of | Baseline | | % epg |
|---|---|---|---|---|
| Treatment | Children | mean epg | % cured | reduction* |
| PZQ | 26 | 979.5 | 30 (8/26) | 81 |
| ARA | 19 | 961.3 | 21 (8/29) | 65 |
| PZQ + ARA | 22 | 805.6 | 68 (15/22) | 84 |

*% epg reduction = ((mean baseline epg − mean epg 6 weeks after treatment)/mean baseline epg) * 100

TABLE 10
Effect of Treatment on IL-10 levels
with PZQ, ARA or PZQ + ARA

| | | | Median | |
|---|---|---|---|---|
| | | Baseline | IL-10 after | % reduction |
| | Number of | median IL-10 | treatment | in IL-10 |
| Treatment | Children | (pg/mL) | (pg/mL) | levels |
| PZQ | 46 | 60 | 36 | 42 |
| ARA | 53 | 80 | 40 | 50 |
| PZQ + ARA | 48 | 70 | 40 | 54 |

TABLE 11
Effect of Treatment on IFN-γ levels
with PZQ, ARA or PZQ + ARA

| | | | Median | |
|---|---|---|---|---|
| | | Baseline | IFN-γ after | % reduction |
| | Number of | median IFN-γ | treatment | in IFN-γ |
| Treatment | Children | (pg/mL) | (pg/mL) | levels |
| PZQ | 46 | 64 | 32.5 | 60 |
| ARA | 53 | 80 | 25 | 70 |
| PZQ + ARA | 48 | 70 | 31 | 56 |

What is claimed is:

1. A composition for the treatment and/or prevention of schistosomiasis, comprising at least one polyunsaturated fatty acid (PUFA) and at least one therapeutic compound.

2. The composition of claim 1, wherein the PUFA is administered in one dosage and the therapeutic compound is administered in a second dosage.

3. The composition according to claim 1 or claim 2, wherein the PUFA is administered in an amount of 5-15 mg/kg body weight.

4. The composition according to any one of claims 1-3, wherein the therapeutic compound is administered in an amount of 20-60 mg/kg body weight/day.

5. The composition according to claim 1, wherein the PUFA and the therapeutic compound are administered in the same dosage.

6. The composition according to claim 5, wherein the PUFA is administered in an amount of 10-600 mg/kg body weight/day.

7. The composition according to claim 5 or claim 6, wherein the therapeutic compound is administered in an amount of 20-60 mg/kg body weight/day.

8. The composition according to any one of claims 1-7, wherein the PUFA is an omega-6 fatty acid.

9. The composition according to claim 8, wherein the PUFA is arachidonic acid.

10. The composition according to any one of claims 1-9, wherein the therapeutic compound is a trematodicide.

11. The composition according to claim 10, wherein the trematodicide is 2-(Cyclohexylcarbonyl)-1,2,3,6,7,11b-hexahydro-4H-pyrazino[2,1-a] isoquinolin-4-one or a derivative thereof.

12. The composition according to any one of claims 1-11, wherein the schistosomiasis is Schistosoma mansoni.

13. A method for treating or preventing schistosomiasis, comprising administering a composition according to any one of claims 1-12.

14. A method for treating and/or preventing schistosomiasis, comprising administering at least one polyunsaturated fatty acid (PUFA) and at least one therapeutic compound in a therapeutically effective amount.

15. The method of claim 14, wherein the PUFA is administered in one dosage and the therapeutic compound is administered in a second dosage.

16. The method according to claim 14 or claim 15, wherein the PUFA is administered in an amount of 5-15 mg/kg body weight.

17. The method according to any one of claims 14-16, wherein the therapeutic compound is administered in an amount of 20-60 mg/kg body weight/day.

18. The method of claim 14, wherein the PUFA and the therapeutic compound are administered in the same dosage.

19. The method of claim 18, wherein the PUFA is administered in an amount of 10-600 mg/kg body weight/day.

20. The method according to claim 18 or claim 19, wherein the therapeutic compound is administered in an amount of 20-60 mg/kg body weight/day.

21. The method of any one of claims 14-20, wherein the PUFA is an omega-6 fatty acid.

22. The method of claim 21, wherein the PUFA is arachidonic acid.

23. The method of any one of claims 14-23, wherein the therapeutic compound is a trematodicide.

24. The method of claim 23, wherein the trematodicide is 2-(Cyclohexylcarbonyl)-1,2,3,6,7,11b-hexahydro-4H-pyrazino[2,1-a]isoquinolin-4-one or a derivative thereof.

25. The method of any one of claims 14-24, wherein the schistosomiasis is Schistosoma mansoni.

26. A method to confer resistance to schistosomiasis, comprising administering at least one polyunsaturated fatty acid (PUFA) and at least one therapeutic compound for treatment and/or prevention of schistosomiasis.

27. The method of claim 26, wherein the PUFA is administered in one dosage and the therapeutic compound is administered in a second dosage.

28. The method of claim 26 or claim 27, wherein the PUFA is administered in an amount of 5-15 mg/kg body weight/day.

29. The method of any one of claims 26-28, wherein the therapeutic compound is administered in an amount of 20-60 mg/kg body weight/day.

30. The method of claim 26, wherein the PUFA and the therapeutic compound are administered in the same dosage.

31. The method of claim 30, wherein the PUFA is administered in an amount of 10-600 mg/kg body weight/day.

32. The method of claim 30 or claim 31, wherein the therapeutic compound is administered in an amount of 20-60 mg/kg body weight/day.

33. The method of any one of claims 26-32, wherein the PUFA is an omega-6 fatty acid.

34. The method of claim 33, wherein the PUFA is arachidonic acid.

35. The method of any one of claims 26-34, wherein the therapeutic compound is a trematodicide.

36. The method of claim 35, wherein the trematodicide is 2-(Cyclohexylcarbonyl)-1,2,3,6,7,11b-hexahydro-4H-pyrazino[2,1-a]isoquinolin-4-one or a derivative thereof.

37. The method of any one of claims 26-36, wherein the schistosomiasis is Schistosoma mansoni.

38. The method of any one of claims 26-37, wherein the levels of IL-10 and IFN-γ in an individual are decreased after administration of the PUFA and the therapeutic compound.

39. A method to prevent re-occurrence of schistosomiasis, comprising administering at least one polyunsaturated fatty acid (PUFA) and at least one therapeutic compound.

40. The method of claim 39, wherein the PUFA is administered in one dosage and the therapeutic compound is administered in a second dosage.

41. The method of claim 39 or claim 40, wherein the PUFA is administered in an amount of 5-15 mg/kg body weight/day.

42. The method of any one of claims 39-41, wherein the therapeutic compound is administered in an amount of 20-60 mg/kg body weight/day.

43. The method of claim 39, wherein the PUFA and the therapeutic compound are administered in the same dosage.

44. The method of claim 43, wherein the PUFA is administered in an amount of 10-600 mg/kg body weight/day.

45. The method of any one of claim 43 or claim 44, wherein the therapeutic compound is administered in an amount of 20-60 mg/kg body weight/day.

46. The method of any one of claims 39-45, wherein the PUFA is an omega-6 fatty acid.

47. The method of any one of claims 39-46, wherein the PUFA is arachidonic acid.

48. The method of any one of claims 39-47, wherein the therapeutic compound is a trematodicide.

49. The method of claim 48, wherein the trematodicide is 2-(Cyclohexylcarbonyl)-1,2,3,6,7,11b-hexahydro-4H-pyrazino[2,1-a]isoquinolin-4-one or a derivative thereof.

50. The method of any one of claims 30-49, wherein the schistosomiasis is Schistosoma mansoni.

51. The method of any one of claims 39-50, wherein the levels of IL-10 and IFN-γ are decreased in an individual after administration of the PUFA and the therapeutic compound.

52. Use of a composition comprising a PUFA or a therapeutic compound for the treatment and/or prevention of schistosomiasis.

53. Use according to claim 52, wherein the PUFA is administered in one dosage and the therapeutic compound is administered in a second dosage.

54. Use according to claim 52 or claim 53, wherein the PUFA is administered in an amount of 5-15 mg/kg body weight.

55. Use according to any one of claims 52-54 wherein the therapeutic compound is administered in an amount of 20-60 mg/kg body weight/day.

56. Use according to any one of claims 52-55, wherein the PUFA and the therapeutic compound are administered in the same dosage.

57. Use according to claim 56, wherein the PUFA is administered in an amount of 10-600 mg/kg body weight/day.

58. Use according to claim 56 or claim 57, wherein the therapeutic compound is administered in an amount of 20-60 mg/kg body weight/day.

59. Use according to any one of claims 52-58, wherein the PUFA is an omega-6 fatty acid.

60. Use according to any one of claims 52-59, wherein the PUFA is arachidonic acid.

61. Use according to any one of claims 52-61, wherein the therapeutic compound is a trematodicide.

62. Use according to claim 61, wherein the trematodicide is 2-(Cyclohexylcarbonyl)-1,2,3,6,7,11b-hexahydro-4H-pyrazino[2,1-a]isoquinolin-4-one or a derivative thereof.

63. Use according to any one of claims 52-62, wherein the schistosomiasis is Schistosoma mansoni.

64. Use of a composition according to any one of claims 52-63 in the manufacture of a medicament for the treatment and/or prevention of schistosomiasis.

65. A composition for the prevention or treatment of schistosomiasis comprising 10 mg/kg body weight/day of a polyunsaturated fatty acid (PUFA) and 40 mg/kg body weight/day of a trematodicide.

66. The composition of claim 65 wherein the PUFA is arachidonic acid.

67. The composition of claim 65 or claim 66 wherein the trematodicde is 2-(Cyclohexylcarbonyl)-1,2,3,6,7,11b-hexahydro-4H-pyrazino[2,1-a] isoquinolin-4-one or a derivative thereof.

68. The composition of any one of claims 64-67 wherein the PUFA is administered in one dosage and the trematodicide is administered in a second dosage.

69. The composition of any one of claims 64-68 wherein the PUFA and the trematodicide are administered in the same dosage.

70. A composition for the prevention of treatment of schistosomiasis comprising 150 mg/kg body weight/day of a polyunsaturated fatty acid (PUFA) and 40 mg/kg body weight/day of a trematodicide.

71. The composition of claim 70 wherein the PUFA is arachidonic acid.

72. The composition of claim 70 or claim 71 wherein the trematodicde is 2-(Cyclohexylcarbonyl)-1,2,3,6,7,11b-hexahydro-4H-pyrazino[2,1-a] isoquinolin-4-one or a derivative thereof.

73. The composition of any one of claims 70-72 wherein the PUFA is administered in one dosage and the trematodicide is administered in a second dosage.

74. The composition of any one of claims 70-72 wherein the PUFA and the trematodicide are administered in the same dosage.
